SEQUENCE LISTING (1 sequence)

SEQ ID NO: 1 - DNA, 5600 bp, homo sapiens
  misc_feature (10)..(10): Location of polymorphism.  Nucleotide can be T or C.
tatgtctcgntttatttactacgaataggtatgatatacatttatctcatgagcctaccttgcttttgtaaagtattttgaaatgtttatgtgaaaatgttgaataggtggtaagaatatggattattgctgtaaattataagcaagagtgaagattgtaactactaaaacactcaattatgtctcaagtgtataaaaatgagaatattttaataaattaaatatcatatatttatgaatcaaagctaaatgttcattaatatcaagtaaatatcagtaaacatgggagatataaaaatgtatgagacaattctggctctcagtgagcttacaatctataagagaagaaaaggggaaaatataaaaattaaataacaacataagaagtatcaaaaggcaacctattgtaaattgccanctgaaatgaaattggcagagtcactactataggtcagaggaaggagagatcattttcagaagcagtgggcagagaagataaagagtccagccacatctccaaggctgaattcgctgggaagcaggaggaggttttggtggagggaacccagtgagaaagtatagatgtacaaaaggaaggagtatggatccccaaaaggaaaaagaatacagaaatgcaagttatggtgataaggagagttctaatatgaaatagggctggaaaggccatttagaattggaccggggagaactgtgaatgttaaattgaagaggtaaggttgctgaacagaaaagcatcttggtcaaagcagcattttggaaatantcctttggttatgatatgccagattaaatggagcagggaggaccagagacagggagatcaaatagaacccatggcaagtcccggtgtggggtgaaggtggggggatgggaaaggaagaagttgagataggaactctgtggaagaagtgcacgacctctcaagtgtgctgatcagccctcattgttggagctttctgtcattttccaaagcattgtgaaatatatgggtttttagaactgactctctttaacagttgccaagtgaagaatgaagaaatgaaggagttttatttaacttacaatatccgatacctgtaaaagttgatacgttgaaccttttcttcttgatcttgtatccttacctaagaataaaaagtgttaggctanaaaaaaagttaaaaaaaaaaaacaccaatcataaaatgtaggaattaaaggagttctgagaacattttatttttattagacaaccaactctagttgaacatgtactattccaaactcttctgtcagtaaaattgaaggaaatcccccagaattcctgagatccctgtactggtactcctgctagaataaccttctggtttcgagggtttataatttaatgtattatacagaaattcggagaaacttgatgtatctctgaatgagcagagaaaaaaagtttcaggaaagttttccaacaattccaatgtcacgatgtgtttatatatataatgatgatacggcaaagttactggcaatctggcttctgccctgaacctctacgaccaagttctggccttatcgcacacagaaaagcaatgccttccacncttcgggggcatttaaggttgacacactctattccggtaacaccacttgacagaaagatggcaaggaaaagcctcttccccacattccctatgcctcaccctgcagctcagcactcagaactcctttcctgggcagcgtcaatttcaacttcttccaatcccttggctattttgagactgccccttgngggtccagaaggaagccaggaggccggggacagtggctcatgcctctaatcccagcactttgggaggccaaggtgggtggatcacctgaggtcaggagttcgagaccagcctgaccaacatggtaaaacgccatctccactaaaaatacaaaattagccgtgtgtggcggcacacgcctgtaatcccagctacttgacaggctgaggcaggagaatcacttgaacccgggaggcagaggttgcagtgggccaagatcacgccactgaactccagcctgggcaacaaaagtgaaactctgtctcaaaaaaataaaaagaaagaagccaggagtctcaggcttncagtgacttggataacttggggtgtagtctccttcctcccctttctaacgcctcttgtttcctggccactcctcctcaacctccccaagttggaaaatcagctgataaatcatgggcagggggtgaataaacaaatcactccccagttttaacanactgatgctgaggtttgggcctaaagcaaattcatatgtttgcatttatgtaagagaaagtggttaaaactgcttgattttatataaacttgaaaaacattactttttgcttttgacctggacagctgtggaagtcggtctctccttgcaaggtaaggatatgagcttggtcatattgatgaccatcaacgaacatgatgttcgttacgggcgctgttcttattccaatgctagaaggacctgtgagcattatataacattttcatgcacccttacataaacaattagaagctgtttactcctcaaggactcaattagagatttttttaatttttttatttttttttttatttcaggcctgagctgaggacactacaggagccaaattagagaaggggcttgcagtcctgccatattgcatattcatggctttcttgtcccctcacccccagtataatcctttttgccaggttaacaactcactaatttcaaagcaattcatttgccaactcaaaaaaaaaaaaaaaaaaaaccacagatacaactttaaatgcttttggattttaatcctctggagtatttatatgttttcttgtctcatcccttcaaaactaatctcagagttttgagaatctgggaacttgggcaaaggagaaaacaagcacgcagaccaaggaatctgaaatcgcagttcattacgtcagcataaactgacagtacctttttgtttgtattgacccagctcaaattataaaatcacatgagtaataaaacacaaaatacaggtgttatcattgaaaagtctaaaatgtaattaaacgcgttttttccccctcgcggtggtatcgtttactctgtatctcaacgtttctgtctccctaagcctctcctttcatatcatgagcatacatttttgcatcatgctcacacgttcattagctaggactgaggagtgtgtacgattccaaagggccctcagcgttagctgttagatgcacaaaccttcgcttcctttccacanctactgcactcgaggttcaacagaggatccttgcaagaacagcgcccagagagcattcttgacagatgcgcgcttggctccagcaacccgctctgctgggaagtttcttctaaccactaacacccacctccaatcccccaagctgtcacgacgcatgctggctgggtcccgtcttgacgggggaagggttttacacacaccctgctaggctgccccacatcacaaccaagctcgcagggcaaactcctccaagcctggcggacaagctgtcccaggcgctctggcgcttcctgaacaccaaggtcccctccccgctcaagggagctagcgctctgttccgcagagaaccccggaactgcaggtcgaggggatgcggggggagcgggggcgcaggagggagccgagtgctggaggcaaacggggcgcaggagcgggtgcgggaggcaaacggggcgcaggagcgggtgcgggaggcaaacggggcgcaggagtgggtgcgggagcgagtgggggctgagggaggggacggggccccggggggagccaggcgcggaagggggcgcgggggaacagggaccaggaaccagcgggcgcaggaaggggngcgtccgcaggaacccgcgggcgcacgggaggcactagctacgcgatcagctcgggactctcaggagccgctcaattgccaacgggaggggggtgcggggagttggaggtgggggtgcagaccagacgggggcgtgcctttgcccggattggctgcaggagcctgacgcgaggccccgggggttggcttggggagtgggagcggggtggggtgggtgctgggtgccggagctgcgggcccggcgcgctcagaaacatgctgaagtcccggcggctcttccagcagcggcagcggctccagcagcagcggcggcggcggcggcggcggcagcggcagcgacagcgctcggctcctgcgggaaaggcgcccggcgcccatgcctccggccccgcgccgcggctgccctgacccggccgcgacctccctctgcgcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaangccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctngtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagnnactgctctgc

(19) United States
(12) Patent Application Publication
(10) Pub. No.: US 2009/0092964 A1
(43) Pub. Date: Apr. 9, 2009

(54) Methods for Individualizing Cardiovascular Disease Treatment Protocols Based on Beta-1 Adrenergic Receptor Haplotype

(75) Inventor: Stephen B. Liggett, Clarksville, MD (US)

Correspondence Address: DINSMORE & SHOHL, LLP, 1900 CHEMED CENTER, 255 EAST FIFTH STREET, CINCINNATI, OH, 45202, US

(73) Assignee: THE UNIVERSITY OF CINCINNATI, Cincinnati, OH (US)

(21) Appl. No.: 12/208,662

(22) Filed: Sep. 11, 2008

Related U.S. Application Data
(60) Provisional application No. 60/971,360, filed on Sep. 11, 2007.

(51) Int. Cl. C12Q 1/68

(52) U.S. Cl. 435/5

(57) ABSTRACT

A method is provided for determining whether a treatment protocol for a human patient who is suffering from heart failure, ischemic heart disease, cardiac arrhythmias, or hypertension includes administration of a beta blocker, the method including obtaining a biological sample from the patient, determining a β₁-adrenergic receptor (β₁AR) sequence of a β₁AR gene from the biological sample, identifying locations of any polymorphisms in the β₁AR sequence, assigning a haplotype to the β₁AR sequence based on the locations identified, and determining whether the treatment protocol includes administration of a beta blocker to the patient based on the haplotype assigned.

### RELATED APPLICATION

This application claims the benefit of U.S. Provisional Application No. 60/971,360, filed Sep. 11, 2007, which application is hereby incorporated by reference in its entirety.

### FIELD OF THE INVENTION

The present invention relates to the fields of pharmacogenetics and cardiology. More specifically, the present invention relates to methods for individualizing cardiovascular disease treatment protocols involving beta adrenergic receptor blocking drugs, based on a patient's β₁-adrenergic receptor (β₁AR) haplotype, as determined by combinations of 15 polymorphisms of the β₁AR gene.

### BACKGROUND OF THE INVENTION

β₁-adrenergic receptors (β₁ARs) are expressed on a number of cell types, including cardiomyocytes, vascular smooth muscle, epithelium, renal juxtaglomerular, and adipocytes. These receptors are targets for agonists in the acute treatment of decompensated heart failure, while β₁AR antagonists are utilized in the treatment of cardiovascular diseases such as chronic heart failure, ischemic heart disease, cardiac arrhythmias, and hypertension, with their major mode of action being antagonism of the β₁AR subtype. However, the response to β₁AR agonists or antagonists appears to be highly variable between individuals, which is not readily explained by clinical status or demographic characteristics. Furthermore, the expression of β₁AR, and the responsiveness to stimulation, can differ substantially between healthy individuals. Such interindividual variability suggests that the receptor may be polymorphic in the population, giving rise to altered expression and physiologic or pharmacologic responsiveness.

Two single nucleotide polymorphisms (SNPs) of β₁AR at nucleotide 145 (A or G, encoding Ser or Gly at amino acid 49) and nucleotide 1165 (G or C, encoding Gly or Arg at amino acid 389) have been previously identified. However, studies involving these two SNPs have produced inconsistent outcomes, suggesting other genetic factors may be involved in gene expression. The need exists to identify other functional polymorphisms in the β₁AR gene outside the coding region and characterize the specific combinations of polymorphisms, or haplotypes.

### SUMMARY OF THE INVENTION

Cardiac β₁-adrenergic receptor (β₁AR) responsiveness in heart failure, ischemic heart disease, cardiac arrhythmias, and hypertension exhibits interindividual variation due to polymorphisms of the intronless β₁AR gene. Analysis of two reference populations yielded data regarding the distribution of specific combinations of polymorphisms (haplotypes) and the effects of those haplotypes on β₁AR expression.

Accordingly, it is an object of the invention to provide methods for individualizing cardiovascular disease treatment protocols involving drugs known as beta blockers, based on a patient's β₁-adrenergic receptor (β₁AR) haplotype, as determined by combinations of 15 polymorphisms of the β₁AR gene.

In one aspect of the invention, a method is provided for determining whether a treatment protocol for a human patient who is suffering from one or more of heart failure, ischemic heart disease, cardiac arrhythmias, or hypertension comprises administration of a beta blocker, the method comprising obtaining a biological sample from the patient, determining a β₁-adrenergic receptor (β₁AR) sequence of a β₁AR gene from the biological sample, identifying locations of any polymorphisms in the β₁AR sequence, assigning a haplotype to the β₁AR sequence based on the locations identified, and determining whether the treatment protocol comprises administration of a beta blocker to the patient based on the haplotype assigned.

In another aspect of the invention, a method is provided for determining a suitable dose of a beta blocker as part of a treatment protocol for a human patient who is suffering from one or more of heart failure, ischemic heart disease, cardiac arrhythmias, or hypertension, the method comprising obtaining a biological sample from the patient, determining a β₁-adrenergic receptor (β₁AR) sequence of a β₁AR gene from the biological sample, identifying locations of any polymorphisms in the β₁AR sequence, assigning a haplotype to the β₁AR sequence based on the locations identified, and determining a suitable dose of the beta blocker as part of the treatment protocol for the patient based on the haplotype assigned.

In another aspect of the invention, a method is provided for determining whether administration of a beta blocker to a patient is indicated or not indicated, the method comprising obtaining a biological sample from the patient, determining a β₁-adrenergic receptor (β₁AR) sequence of a β₁AR gene from the biological sample, identifying locations of any polymorphisms in the β₁AR sequence, assigning a haplotype to the β₁AR sequence based on the locations identified, and determining that administration of a beta blocker is indicated or not indicated based on the haplotype assigned.

In still another aspect of the invention, a screening assay is provided for evaluating the effectiveness of a beta blocker against one or more target β₁AR haplotypes comprising (a) establishing baseline expression levels for cells expressing one or more target β₁AR haplotypes, (b) exposing cells that express one or more target β₁AR haplotypes to a beta blocker, (c) determining expression levels of the one or more target β₁AR haplotypes, (d) comparing the expression levels of step (c) with the baseline expression levels, and (e) evaluating the effectiveness of the beta blocker against the one or more target β₁AR haplotypes based on the comparison of step (d).

### BRIEF DESCRIPTION OF THE DRAWINGS

FIG. 1. Localization of sequence variants within the β₁AR gene. Shown are all variants detected, with those annotated by the asterisk having allele frequencies >=0.05 in at least one of the two reference populations.

FIG. 2. Differential expression of β₁AR haplotypes. The intronless β₁AR gene was amplified from genomic DNA, and haplotypes constructed and subcloned into the promoterless vector pCR2.1. A431 cells were transfected; 48 hours later, membranes were prepared, and quantitative radioligand binding with ¹²⁵I-CYP was carried out. *Expression greater than all other haplotypes (P<0.05) but not different from each other; +expression lower than all other haplotypes (P<0.01); #expression not different from each other but lower than haplotypes 2 and 4 and higher than haplotype 6 (P<0.05). Endogenous expression of β₁AR after mock transfections amounted to 16±7.1 fmol/mg. Results are from eight independent experiments.

FIG. 3. β₁AR haplotype clustering based on expression phenotypes. The column and row headings represent the β₁AR haplotypes. The values in each cell are ratios of the row haplotype expression/column haplotype expression, such that values <1 represent low expression relative to the given haplotype, and values >1 represent high expression. Haplotypes with highlights in light grey indicate high expression; dark grey, low expression; and white, intermediate expression.

### DETAILED DESCRIPTION OF THE INVENTION

The following terms are used in the present application:

The term “polymorphism” is used herein to refer to genetic variations of a nucleic acid sequence occurring in a statistically significant percentage of a population of subjects. In the present invention, 15 polymorphisms within the 6.1 kb β₁AR gene with allele frequencies ≧0.05 were identified in the 5′-flanking and coding regions. The relevant 5.6 kb portion of the sequence is provided herein as SEQ. ID NO. 1. These 15 polymorphisms and their frequencies in the test populations (white of European Descent (ED), or black of African Descent (AD)) are described in the following Table 1. Nucleotide positions are indicated relative to the ATG start codon of the β₁AR gene, where A in the ATG start codon is designated as position +1, and are also indicated relative to the nucleotides numbers as provided herein in SEQ. ID NO. 1.

TABLE 1
Common polymorphisms of the β₁AR gene.

| | Nucleotide | Nucleotide | | Minor Allele | | |
|---|---|---|---|---|---|---|
| | relative to A of | relative to | Alleles | Frequency (%) | | |
| Region | ATG start codon | SEQ. ID NO. 1 | (major/minor) | AD | ED | All |
| Promoter | −4416 | 10 | tctcgT/Ctttatt | 20 | 35 | 28 |
| | −4266* | 159** | gattgtDEL/INSaactac | 6 | 0 | 3 |
| | −4007 | 418 | ttgccaC/Tctgaaa | 6 | 0 | 3 |
| | −3641 | 784 | gaaataC/Ttccttt | 4 | 22 | 13 |
| | −3255 | 1170 | aggctaA/Caaaaaa | 4 | 21 | 12 |
| | −2827 | 1598 | ttccacC/Acttcgg | 0 | 7 | 3 |
| | −2639 | 1786 | cccttgT/Cgggtcc | 32 | 39 | 35 |
| | −2297 | 2128 | aggcttT/Gcagtga | 21 | 23 | 22 |
| | −2142 | 2283 | ttaacaT/Cactgat | 5 | 23 | 14 |
| | −1121 | 3304 | tccacaT/Cctactg | 5 | 0 | 3 |
| | −517 | 3908 | aaggggT/Cgcgtcc | 14 | 18 | 16 |
| Coding | +145 | 4569 | agcgaaA/Ggccccg | 15 | 22 | 18 |
| | +315 | 4739 | cgacctG/Tgtcatg | 15 | 0 | 8 |
| | +1165 | 5589 | ttccagC/Ggactgc | 53 | 28 | 38 |
| | +1166 | 5590 | tccagcG/Tactgct | 7 | 0 | 3 |

*At position −4266, the insertion is an A
**At position 159, the insertion is an A
AD, African-descent; ED, European-descent

In describing the location of the polymorphic sites identified herein, reference is made to the sense strand of the gene for convenience. However, as recognized by the skilled artisan, nucleic acid molecules containing the β₁AR gene may be complementary double stranded molecules and thus reference to a particular site on the sense strand refers as well to the corresponding site-on the complementary antisense strand.

These common polymorphisms are organized by the present invention into six haplotypes. The term “haplotype” is used herein to refer to different combinations of 15 polymorphisms of the β₁AR gene that have been found in the general population. As used herein, the terms “haplotype 1”, “haplotype 2”, “haplotype 3”, “haplotype 4”, “haplotype 5”, and “haplotype 6” refer to the combinations of 15 polymorphisms of the β₁AR gene, as set forth in the following Table 2, wherein the locations of the polymorphisms are given relative to the ATG start codon of the β₁AR gene and relative to the nucleotide numbers in SEQ. ID NO. 1:

TABLE 2

_{β1}AR haplotypes with frequencies ≧5%.

Location*

| Haplotype | −4416 | −4266 | −4007 | −3641 | −3255 | −2827 | −2639 | −2297 | −2142 |
|---|---|---|---|---|---|---|---|---|---|
| No | (10) | (159) | (418) | (784) | (1170) | (1598) | (1786) | (2128) | (2283) |
| 1 | T | D | C | C | A | C | T | T | T |
| 2 | T | D | C | C | A | C | C | T | T |
| 3 | C | D | C | T | C | C | T | G | C |
| 4 | C | D | C | C | A | C | T | T | T |
| 5 | T | D | C | C | A | C | C | T | T |
| 6 | T | D | C | C | A | A | T | T | T |

Location*

| | | | | | | | | |
|---|---|---|---|---|---|---|---|---|
| | | | | | | | Minor | Minor |
| | | | | | | | Allele | Allele |
| Haplotype | −1121 | −517 | +415 | +315 | +1165 | +1166 | Frequency | Frequency |
| No | (3304) | (3908) | (4569) | (4739) | (5589) | (5590) | (%) AD | (%) ED |

| | | | | | | | | |
|---|---|---|---|---|---|---|---|---|
| 1 | T | T | A | G | G | G | 41.7 | 20.8 |
| 2 | T | T | A | G | C | G | 16.7 | 35.4 |
| 3 | T | C | G | G | C | G | 4.2 | 20.8 |
| 4 | T | T | A | G | C | G | 4.2 | 12.5 |
| 5 | T | T | A | T | C | G | 6.3 | 0 |
| 6 | T | T | A | G | G | G | 0 | 6.3 |

D = deletion (see Table 1)
AD, African-descent; ED, European-descent
*the first number represents location relative to the A of the ATG start codon. The number in parenthesis is relative to SEQ. ID NO. 1.

The term “expression” refers to the production of a functional gene product from a gene. The product of expression may be a peptide, protein, or nucleotide sequence in a cell, such as an RNA product. Products of expression may be obtained from cells and/or cell culture media, as well as from biological samples obtained from test subjects, such as humans or other animals. “Expression levels” refers to a quantitative measurement of a gene product. “Baseline expression levels” are expression levels which are suitable for use as controls in assessing the change in expression levels due to experimental factors. In one embodiment of the present invention, baseline expression levels are established in a screening assay prior to exposing cells to a test compound, or potential beta blocker.

“Biological sample” refers to any sample obtained from a subject that contains genetic material that can be extracted to provide genomic information relating to the subject (including, but not limited to, DNA, RNA, proteins, antibodies, and the like). The sample may, for example, comprise blood, buccal swab, hair, paraffin-embedded tissue, biopsy tissue, or any other biological sample from which genetic material may be extracted. In one embodiment of the invention, the sample comprises genomic DNA capable of extraction and purification.

“Cardiovascular disease” refers to the class of diseases that involves the heart or blood vessels. As used herein, the term “cardiovascular disease” includes, but is not limited to, heart failure, hypertension, ischemic heart disease (including coronary artery disease, myocardial infarction, and angina) and cardiac arrhythmias.

The term “heart failure” is used herein to refer to the physiological state in which cardiac output is insufficient for the body's needs. Heart failure may occur when the structure or function of the heart impairs its ability to provide sufficient blood flow to maintain the body.

The term “hypertension” used herein refers to symptoms related to undesirably high levels of blood pressure. Individuals said to have symptoms related to hypertension have blood pressure levels at an undesirably high level. For example, an individual with a diastolic blood pressure above 89 mmHg and a systolic blood pressure above 139 mmHg, is considered to have an undesirably high level of blood pressure by the medical community.

“Ischemic heart disease” is used herein to refer to disease characterized by the reduced blood supply to the heart muscle. Examples of ischemic heart disease include coronary artery disease, myocardial infarction, and angina.

“Cardiac arrhythmias” is used herein to refer to a group of conditions in which there is abnormal electrical activity in the heart. The heart beat may be too fast or too slow, and may be regular or irregular.

The term “beta blocker” is used herein to refer to beta-adrenergic receptor blocking agents, i.e., drugs that block sympathetic effects on the heart or other organs such as the kidney and are generally most effective in lowering arterial blood pressure when there is increased sympathetic nerve activity. In addition, these drugs block the adrenergic nerve-mediated release of rennin from the renal juxtaglomerular cells. Beta blockers include the class of drugs known to bind to the β₁- and/or β₂-adrenergic receptors and may act as antagonists, inverse agonists, or weak partial agonists. Examples of this class of drugs include, but are not limited to, metoprolol, carvedilol, bucindolol, atenolol, bisoprolol, betaxolol, timolol, mebivolol, pindolol, propranolol and labetalol.

The term “beta agonist” is used herein to refer to agents that stimulate beta-receptors in the autonomic nervous system. Beta agonists (β-agonists) bind to β-receptors on cardiac and smooth muscle tissues. They also bind to β-receptors in other tissues including bronchial smooth muscle, the liver and kidneys. β-agonists include the class of drugs known to bind to the β₁- and/or β₂-adrenergic receptors and act as agonists. Examples of this class of drugs include, but are not limited to, epinephrine, norepinephrine, dopamine, dobutamine, and isoproterenol.

The term “recommended dose” is used herein to refer to the dose or range of doses of a drug customarily prescribed for or administered to an adult patient from the general population suffering from a particular disease or condition. With respect to beta blockers approved for administration to patients, the recommended dose is dependent upon multiple factors, such as the type, strength, and form of the beta blocker and the condition for which it is prescribed. One skilled in the art, such as a physician, clinician, or pharmacist, will appreciate that different recommended doses may be indicated, depending on the particular beta blocker prescribed and the condition to be treated.

### Methods

### Identification of Sequence Variants and Haplotypes

Sequence variations in the human β₁AR gene (also known by the gene name ADRB1) were identified in silico from the Seattle SNPs database (http://pga.gs.washington.edu), which provided β₁AR sequence data derived from 47 individuals. Race was self-reported and is denoted as white of European descent (ED), or black of African descent (AD). The contiguous sequence that was considered in this intronless gene was from −4500 (relative to the initiator ATG of the coding region), through the 1434-bp coding region, and the complete 3′UTR (203 bp) up to the poly-A termination sequence (approximately to nucleotide +1650). Haplotypes were imputed from these SNPs by the Bayesian method of Donnelly and colleagues, using the program PHASE 10 and assuming a stepwise mutation mechanism. See, e.g., Stephens et al., A new statistical method for haplotype reconstruction from population data. Am. J. Hum. Genet. 2001, 68:978-89. Haplotypes with confidence probabilities >90% were considered for further evaluation. Resequencing of selected regions from specific genomic DNA samples from the human variation panel of the Coriell Institute (http://ccr.coriell.org/nigms/cells/humdiv.html ) identified from the Seattle SNPs database were carried out with an ABI Prism 3700 Sequencer (Applied Biosystems) using dye terminator chemistry to confirm some haplotypes.

### Mutagenesis and Haplotype Construction

Reference sequences (e.g., accession no. NM_{—}000684, last accessed Sep. 9, 2008) were used to design polymerase chain reactions (PCRs) to amplify multiple fragments of the β₁AR gene from specific human genomic DNA samples derived from the human variation panel. These constructs were then digested with restriction enzymes and were ligated to build the β₁AR haplotypes. In selected cases, site-directed mutagenesis was used to generate specific base changes, using previously described methods. Mason D A, et al. A gain-of-function polymorphism in a G-protein coupling domain of the human β₁-adrenergic receptor. J. Biol. Chem. 1999, 274:12670-74. The final constructs used for transfections consisted of contiguous β₁AR sequence from −4465 to +1637 with the indicated sequence variants in the expression vector pCR2.1 (Invitrogen).

### Transfection and Cell Culture

The human epithelial carcinoma cell line A431 (American Type Culture Collection, Manassas, Va.) was grown as monolayers at 37° C. in a 95% air 5% CO₂ atmosphere, in Dulbecco's modified Eagle's medium with 4 mM L-glutamate, 10% fetal bovine serum, 100 U/mL of penicillin, and 100 μg/mL of streptomycin. Cells at 50% confluency were incubated with 30 μL of Lipofectamine 2000 (Invitrogen, Carlsbad, Calif.), 15 μg of β₁AR construct (or empty vector), and 8 mL of serum-free media for 6 hours at 37° C. Subsequently, additional media was added to bring the serum concentration to 10%, and the cells were maintained for 12 hours. After a change to fresh media with 10% serum, cells were incubated an additional 24 hours. These conditions provided for β₁AR protein expression (as determined by quantitative radioligand binding) within the linear range of the transfected DNA:expression curve.

### Radioligand Binding

Transfected cells were washed four times with PBS at 25° C. and lysed in 10 mL of 5 mM Tris and 2 mM EDTA buffer (pH 7.40) at 4° C., detached with a cell scraper, and sheared by repeated pipetting. The preparation was centrifuged at 30,000 g for 15 minutes at 4° C., and the pellet was resuspended in 75 mM Tris, 12 mM MgCl₂, 2 mM EDTA, pH 7.40. Quantitative radioligand binding with the β₁AR antagonist ¹²⁵I-cyanopindolol (¹²⁵I-CYP, PerkinElmer, Waltham, Mass.) was carried out in triplicate, as previously described (Mason et al.). Nonspecific binding was defined with 10 μM propranolol. Specific binding is expressed as femtomoles (fmol) per milligram of protein.

### Statistical Analysis

Data are presented as means±standard errors. Allele frequencies were calculated using standard gene-counting methods. The confidence probabilities of the imputed haplotypes were calculated as described. Stephens M, et al., A new statistical method for haplotype reconstruction from population data. Am. J. Hum. Genet. 2001, 68:978-89. Statistical comparisons of expression were performed by ANOVA, and, using post hoc Student's t tests, the haplotypes with expression levels that did not differ were grouped. These groups were then compared, with significance considered when P<0.05.

### Results

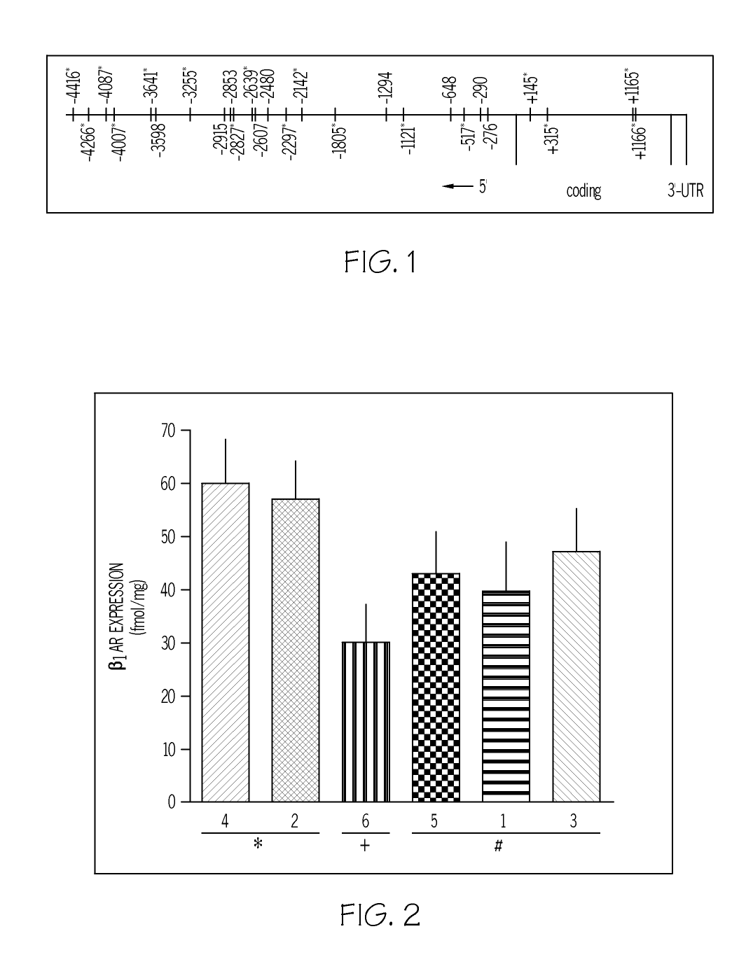

The locations within the β₁AR gene of all variants that were found in the two ethnic groups are depicted in FIG. 1. Those variants with allele frequencies >0.05 (indicated by the asterisk in FIG. 1) in either reference population were considered for further study, and their flanking sequences, allele frequencies, and ethnic distributions are shown in Table 1. Altogether, 15 variable loci fit this criteria: 11 were in the 5′-flanking region, and 3 nonsynonymous and 1 synonymous were within the coding block. No variants were found in the 203 bases of 3′UTR leading to the termination sequence. All were SNPs, except for the uncommon insertion at −4266. Several SNPs were found in only one of the two ethnic groups, five being found exclusively in the AD (−4266, −4007, −1121, 315, and 1166), and one in the ED population (−2827). The remaining nine SNPs were cosmopolitan, although several-fold differences in allele frequencies between the two ethnic groups were noted for SNPs at positions −3641, −3255, −2142, and +1165.

The β₁AR polymorphisms were found to be organized into six haplotypes with allele frequencies >0.05 in one of the two ethnic groups (Table 2). Two ethnic-specific haplotypes (haplotype 5 in AD and haplotype 6 in ED) were noted. The other four haplotypes were found in both ethnic groups, although two- to fivefold differences in frequency were noted between ethnic groups. Haplotype 1 was by far the most common AD haplotype, with a frequency greater than twofold that of the next most common haplotype in this population. In contrast, haplotypes 2, 3, and 4 were relatively common in the ED population, with haplotype 2 being most prevalent.

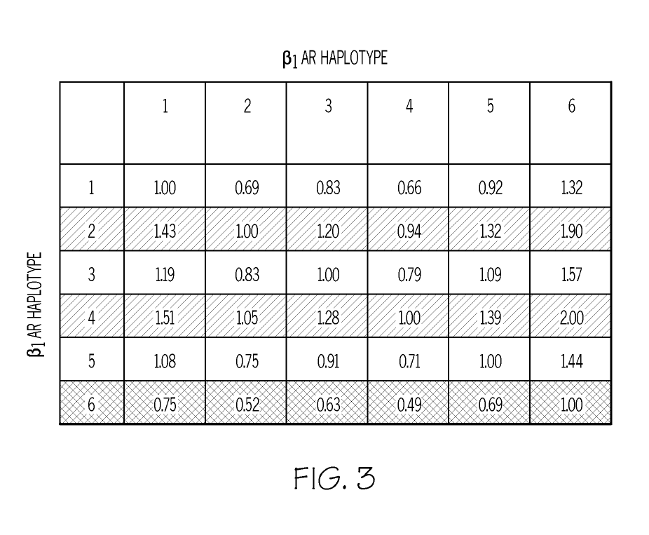

To ascertain potential consequences of these variations on receptor protein expression, whole gene transfections were carried out with A431 cells. This immortalized cell line was used because it is of human origin, and it expresses the β₁AR (at approximately 15 fmol/mg) and, thus, the necessary transcription factors to promote expression in a gene-specific manner. The constructs consisted of the contiguous DNA sequence (from −4465 to +1637) representing haplotypes with allele frequencies >0.05 (5%) in either ethnic group, subcloned into the vector pCR2.1. This vector lacks a eukaryotic promoter, so expression is driven by the β₁AR promoter itself, and thus the impact of variations can be assessed. β₁AR protein expression was determined by quantitative radioligand binding with [¹²⁵I]CYP. The results are shown in FIG. 2, expressed as fmol/mg protein. A relationship between haplotype and expression was noted at P=0.006 by ANOVA, but it is clear that some haplotypes have very similar expression levels. The expressions of any one haplotype compared with any other haplotype are provided as ratios in matrix form in FIG. 3. As can be seen, the expression phenotypes are found in three clusters. Haplotypes 2 and 4 represent a high expression phenotype, with greater expression (FIG. 3 ratios >1.0, light grey bars) than all other haplotypes. Haplotype 6 was consistently expressed lower than the other haplotypes (FIG. 3 ratios <1.0, dark grey bar). Intermediate expression was found with haplotypes 1, 3, and 5.

### Discussion

As introduced earlier, β₁ARs are expressed on a number of diverse cell-types, including cardiac myocytes. The goal of the current work was to identify the common genetic variants of this gene, ascertain the haplotypes that exist in the general population, and determine their expression phenotypes. The ability to define this phenotype is possible because the gene is intronless and of relative small size, which facilitates whole-gene transfections. Furthermore, we used expression of the receptor itself, rather than a reporter gene approach, the latter not providing for interactions between promoter and coding polymorphisms or the influence of the 3′UTR. We identified six haplotypes with frequencies >0.05 in at least one of the two reference groups. Although there are also low-frequency polymorphisms and, thus, haplotypes (the number of which increases as the number of interrogated subject genomes increases), the sample size used for polymorphism discovery provides for an expected detection rate of 99% of the polymorphisms with allele frequencies >=0.05. In the expression studies, we found a significant relationship between β₁AR haplotype and receptor expression. Furthermore, the expressions seemed to lie within three groups: haplotypes 2+4, haplotypes 1+3+5, and haplotype 6.

Our results have potential implications for a number of diseases, including hypertension, ischemic heart disease, cardiac arrhythmias, and heart failure. Of particular interest is the β₁-Arg/Gly polymorphism at amino acid 389, which has been associated with multiple heart failure phenotypes, although there are a number of discrepant reports from different investigators. See, e.g., Liggett S B, Genetic, molecular, and clinical characterization of adrenergic receptor polymorphisms., in: Perez D, ed., The Adrenergic Receptors: In the 21st Century. Humana Press, Inc., 2005:339-64; Small K M, et al., Adrenergic receptor polymorphisms in heart failure; molecular and physiologic phenotypes, in: Walsh R A, ed., Molecular Mechanisms of Cardiac Hypertrophy and Failure. Taylor & Francis, 2005:611-24. One explanation for such discrepancies may be additional variability in the measured phenotypes in one or both groups of subjects, when stratified by this single polymorphic locus, because of other β₁AR polymorphisms. Although a nonsynonymous polymorphism at amino acid 49 is also recognized, its frequency is relatively low (homozygosity [almost equal to] 3%) and is unlikely to be the basis for such variability. This prompted the current study to ascertain additional loci of variability outside the coding block, and to consider their relevance in terms of haplotypes. And, indeed, six haplotypes with frequencies >5% were identified. In the ED population, these represent >95% of all haplotypes, whereas in the AD population there are a number of very-low-frequency haplotypes, such that the major six haplotypes represent approximately 75% of all haplotypes found. This greater diversity in AD is not unexpected, given the greater time for the African population to undergo additional events. Expression of the haplotypes varied by as much as twofold. There is a direct correlation between endogenous β₁AR-expression levels and agonist-promoted function in the human heart. Thus, a twofold difference in expression attributable to the genetic variation of the haplotypes would be expected to have an impact on human in vivo responses. The clustering of the expression levels provides an additional analytical tool in association studies. Stratification by haplotype may reduce statistical power because of the small numbers of subjects in six groups. Assignment of subjects to a β₁AR haplotype expression cluster will provide for a smaller number of groups (three) and is biologically justified on the basis of our current results.

### Conclusions

Polymorphisms were identified within the human β₁AR gene and were found to be present within the 5′-flanking and coding regions, but not the 3′UTR. These polymorphisms were characterized as they appear in phase with each other, as haplotypes, by in vitro transfection studies. β₁AR density was affected by these variations, indicating a mechanism by which diversification of receptor expression in the population is attained via genetic means in a haplotype-directed manner.

In one embodiment of the present invention, a method is provided for determining whether a treatment protocol for a human patient who is suffering from one or more of heart failure, ischemic heart disease, cardiac arrhythmias, or hypertension comprises administration of a beta blocker, the method comprising:
(a) obtaining a biological sample from the patient;
(b) determining a β₁-adrenergic receptor (β₁AR) sequence of a β₁AR gene from the biological sample;
(c) identifying locations of any polymorphisms in the β₁AR sequence;
(d) assigning a haplotype to the β₁AR sequence based on the locations identified; and
(e) determining whether the treatment protocol comprises administration of a beta blocker to the patient based on the haplotype assigned.
   In one aspect of this embodiment, the haplotype is selected from the group consisting of haplotype 1, haplotype 2, haplotype 3, haplotype 4, haplotype 5, and haplotype 6. In another aspect of this embodiment, administration of a beta blocker is indicated in the treatment protocol for a patient having haplotype 2 or 4, since those haplotypes have the highest expression levels of β₁AR compared to haplotypes 1, 3, 5 and 6, and would predispose a patient to a favorable response to a treatment protocol including a beta blocker. In another aspect of this embodiment, administration of a beta blocker is not indicated in the treatment protocol for a patient having haplotype 1, 3, 5, or 6, since these haplotypes are lower expressors (compared to haplotypes 2 and 4) and would be predisposed to a lack of response to a treatment protocol including a beta blocker.

One skilled in the art will understand that the biological sample can be any sample comprising genetic material capable of extraction, purification, and sequencing. In one aspect of this embodiment, the biological sample is blood, buccal swab, hair, tissue biopsy, or paraffin-embedded tissue. In another aspect of the invention, the biological sample comprises genomic DNA. Methods for extracting genomic DNA from a biological sample are known in the art and would be apparent to the skilled artisan.

See, for example, Genomic DNA Purification: Technical hints, applications, and protocols, published by Qiagen, 2002, available at http://wwwl.qiagen.com/literature/brochures/Gen_DNA_Pur/1019469_BROS_DNYTi_INT_{—}0502.pdf (Sep. 10, 2008).

Similarly, one skilled in the art will appreciate the variety of beta blockers available for inclusion in treatment protocols and will be able to select the appropriate beta blocker for the condition to be treated, provided treatment with a beta blocker is indicated for the patient's haplotype. In one aspect of this embodiment, the beta blocker is selected from the group consisting of metoprolol, carvedilol, bucindolol, atenolol, bisoprolol, betaxolol, timolol, mebivolol, pindolol, propranolol and labetalol. However, one skilled in the art will appreciate that another beta blocker may be appropriate for the patient's needs.

In one aspect of this embodiment, the β₁AR sequence is determined from DNA or RNA in the biological sample. In the case of DNA, various techniques for sequencing are known in the art, and would be apparent to the skilled artisan. See, for example, Current Protocols in Molecular Biology, 2007, John Wiley and Sons, Inc. (Red Book). In one aspect of the invention, RNA can be extracted from the biological sample, translated into DNA using known methods such as reverse transcriptase, providing DNA suitable for sequencing.

In another embodiment of the present invention, a method is provided for determining a suitable dose of a beta blocker as part of a treatment protocol for a human patient who is suffering from one or more of heart failure, ischemic heart disease, cardiac arrhythmias, or hypertension, the method comprising:
(a) obtaining a biological sample from the patient;
(b) determining a β₁-adrenergic receptor (β₁AR) sequence of a β₁FAR gene from the biological sample;
(c) identifying locations of any polymorphisms in the β₁AR sequence;
(d) assigning a haplotype to the β₁AR sequence based on the locations identified; and
(e) determining a suitable dose of the beta blocker as part of the treatment protocol for the patient based on the haplotype assigned.

In one aspect of the embodiment, the haplotype is selected from the group consisting of haplotype 1, haplotype 2, haplotype 3, haplotype 4, haplotype 5, and haplotype 6. In another aspect of the embodiment, the suitable dose of a beta blocker for a patient having haplotype 2 or 4 is lower than a recommended dose, since patients having haplotypes 2 or 4 are predisposed to a more favorable response to treatment protocols including a beta blocker, and are more likely to respond effectively to treatments at doses lower than generally recommended doses. In another aspect of the embodiment, the suitable dose of a beta blocker for a patient having haplotype 1, 3, 5, or 6 is higher than a recommended dose, since patients having haplotypes 1, 3, 5, or 6 are predisposed to a less effective response to treatment protocols including a beta blocker, and are more likely to respond favorably to treatments at doses higher than generally recommended doses.

One skilled in the art will understand that the biological sample can be any sample comprising genetic material capable of extraction, purification, and sequencing. In one aspect of this embodiment, the biological sample is blood, buccal swab, hair, tissue biopsy, or paraffin-embedded tissue. In another aspect of the invention, the biological sample comprises genomic DNA. Methods for extracting genomic DNA from a biological sample are known in the art and would be apparent to the skilled artisan.

Similarly, one skilled in the art will appreciate the variety of beta blockers available for inclusion in treatment protocols and will be able to select the appropriate beta blocker for the condition to be treated, provided treatment with a beta blocker is indicated for the patient's haplotype. In one aspect of this embodiment, the beta blocker is selected from the group consisting of metoprolol, carvedilol, bucindolol, atenolol, bisoprolol, betaxolol, timolol, mebivolol, pindolol, propranolol and labetalol. However, one skilled in the art will appreciate that another beta blocker may be appropriate for the patient's needs.

In another embodiment of the present invention, a method is provided for determining whether administration of a beta blocker to a patient is indicated or not indicated, the method comprising:
(a) obtaining a biological sample from the patient;
(b) determining a β₁-adrenergic receptor (β₁AR) sequence of a β₁AR gene from the biological sample;
(c) identifying locations of any polymorphisms in the β₁AR sequence;
(d) assigning a haplotype to the β₁AR sequence based on the locations identified; and
(e) determining that administration of a beta blocker is indicated or not indicated based on the haplotype assigned.

In one aspect of the embodiment, the haplotype is selected from the group consisting of haplotype 1, haplotype 2, haplotype 3, haplotype 4, haplotype 5, and haplotype 6. In another aspect of the present embodiment, administration of a beta blocker is indicated in a patient having haplotype 2 or 4, since patients having those haplotypes are predisposed to a favorable response to treatment protocols including administration of a beta blocker. In another aspect of the embodiment, administration of a beta blocker is not indicated in a patient having haplotype 1, 3, 5, or 6, since patients with these haplotypes are predisposed to a lack of a response to treatment protocols including administration of a beta blocker.

One skilled in the art will understand that the biological sample can be any sample comprising genetic material capable of extraction, purification, and sequencing. In one aspect of this embodiment, the biological sample is blood, buccal swab, hair, tissue biopsy, or paraffin-embedded tissue. In another aspect of the invention, the biological sample comprises genomic DNA. Methods for extracting genomic DNA from a biological sample are known in the art and would be apparent to the skilled artisan.

In another aspect of the present embodiment, the method further comprises selecting candidate human subjects for participation in a clinical trial involving a beta blocker based on the determination that administration of a beta blocker is indicated or not indicated based on the haplotype assigned. In one aspect of this embodiment, it may be advantageous to select clinical trial participants who are predisposed to a particular response to a beta blocker. In one aspect of the present embodiment, clinicians may select candidates have haplotypes selected from the group consisting of haplotype 1, haplotype 2, haplotype 3, haplotype 4, haplotype 5, haplotype 6, and combinations thereof. In another aspect of the embodiment, clinicians may select candidates having haplotypes 2 or 4, with the understanding that those candidates are predisposed to a favorable response to a beta blocker. In another aspect of the embodiment, a clinician may select candidates having haplotypes 1, 3, 5, or 6, with the understanding that those candidates are predisposed to a less favorable response to a beta blocker, as compared with haplotypes 2 or 4. One skilled in the art would be able to select for the desired haplotype, depending on the specific goals of the clinical trial at issue.

In another embodiment of the present invention, a screening assay is provided for evaluating the effectiveness of a beta blocker against one or more target β₁AR haplotypes comprising:
(a) establishing baseline expression levels for cells expressing one or more target β₁AR haplotypes;
(b) exposing cells that express one or more target β₁AR haplotypes to a beta blocker;
(c) determining expression levels of the one or more target β₁AR haplotypes;
(d) comparing the expression levels of step (c) with the baseline expression levels; and
(e) evaluating the effectiveness of the beta blocker against the one or more target β₁AR haplotypes based on the comparison of step (d).

In one aspect of the embodiment, the one or more target β₁FAR haplotype is selected from the group consisting of haplotype 1, haplotype 2, haplotype 3, haplotype 4, haplotype 5, haplotype 6, and combinations thereof.

In another aspect of the present embodiment, the screening assay is conducted in vitro, in vivo, or ex vivo. In one aspect of the embodiment, the cells of the screening assay may endogenously express the one or more target β₁AR haplotypes. In another aspect, the cells are engineered to express the one or more target β₁AR haplotypes.

“Exposing cells” is used herein to refer to any method by which cells capable of expressing β₁AR are placed in contact with a test compound, such as a beta blocker. In one aspect of the embodiment, the screening assay is an in vitro assay wherein the cells are incubated with a test compound for an appropriate period of time. The time period may be 24 hours, 48 hours, or any other time period appropriate to the assay, as determined by the skilled artisan. In another aspect of the embodiment, the cells are part of a living organism, and the exposing is accomplished by dosing the organism with a test compound. In another aspect of the embodiment, the assay is performed ex vivo on tissue removed from a living organism.

In another embodiment of the invention, a screening assay is provided for evaluating the effectiveness of a beta blocker against one or more target β₁AR haplotypes comprising (a) establishing baseline response to a beta agonist for cells expressing one or more target β₁AR haplotypes, (b) exposing cells that express one or more target β₁AR haplotypes to a beta blocker, (c) determining the response to a beta blocker of the one or more target β₁AR haplotypes, (d) comparing the response of step (c) with the baseline response, and (e) evaluating the effectiveness of the beta blocker against one or more target β₁AR haplotypes based on the comparison of step (d).

As used herein, “response” refers to a physiological change effected in the cells. In one aspect of the embodiment, the assay is conducted in vitro and the response is assessed by measuring, for example, intracellular cAMP. In another aspect of the embodiment, the assay is conducted in vivo or ex vivo, and the response is assessed by measuring, for example, blood pressure, heart rate, and/or cardiac contractility.

In another embodiment of the invention, an in vitro screening assay is provided for determining the capacity of a potential beta blocker to antagonize the cAMP-stimulating effects of norepinephrine or another beta-agonist on one or more of the β₁AR haplotypes.

In still another embodiment of the invention, an in vitro screening assay is provided for determining the affinity of a beta blocker for binding to a β₁AR haplotype.

In still another embodiment of the invention, an in vivo screening assay is provided for determining the effects of a beta blocker on blood pressure, heart rate, and protection against, or improvement in, heart failure induced by transaortic constriction or beta-agonist infusion.

In another embodiment of the invention, an in vivo screening assay is provided for determining the capacity of a beta blocker to antagonize an increase in heart rate by norepinephrine or another beta-agonist.

### EXAMPLES

The following non-limiting examples illustrate the methods and screening assays of the present invention.

### Example 1

A blood sample is obtained from a patient in need of treatment for heart failure. Genomic DNA is isolated from leukocytes in the blood sample using phenol/chloroform precipitation techniques known in the art. The genomic DNA is used as a template for amplification of the β₁AR gene or a fragment thereof by polymerase chain reaction (PCR) covering the nucleotides where there are polymorphisms that define the haplotypes. The PCR product is purified and sequenced using an automated DNA sequencer. The nucleotides at each polymorphic site are identified and the haplotype is noted to be Haplotype 2. Bucindolol, a beta blocker, is prescribed to treat the heart failure because the patient has a haplotype that predisposes to a favorable response to a treatment protocol including a beta blocker.

### Example 2

A patient with heart failure is receiving the beta blocker metoprolol but has complaints of frequent dizzy spells upon standing from a seated or lying position. The patient's heart rate is found to be 55 in both the lying and standing positions, and the systolic and diastolic blood pressures decrease upon standing. The physician is concerned that the dizzy spells are due to the beta blocker, but also is cognizant that administration of a beta blocker is a recognized treatment for heart failure. A blood sample is obtained from the patient. Genomic DNA is isolated from leukocytes in the blood using standard phenol/chloroform precipitation techniques. The genomic DNA is used as a template for PCR amplification of the β₁AR gene or a fragment thereof covering the nucleotides where there are polymorphisms that define the haplotypes. The PCR product is purified and sequenced using an automated DNA sequencer and techniques known in the art. The nucleotides at each polymorphic site are identified and the haplotype is noted to be Haplotype 6. Because the patient has a β₁AR haplotype that corresponds to a low probability that a beta blocker will have a significant therapeutic effect for heart failure in the individual, and because the beta blocker is the likely cause of the patient's dizzy spells in this instance, the patient is withdrawn from the beta-blocker metoprolol.

### Example 3

A buccal swab is obtained from a patient in need of treatment for hypertension. RNA is isolated from the cells in the buccal swap sample. The RNA is translated into DNA using reverse transcriptase and methods commonly used in the art. The DNA is used as a template for PCR amplification of the β₁AR gene or a fragment thereof covering the nucleotides where there are polymorphisms that define the haplotypes. The PCR product is purified and sequenced using an automated DNA sequencer. The nucleotides at each polymorphic site are identified and the haplotype is noted to be Haplotype 4. Atenolol, a beta blocker, is prescribed to treat the patient's hypertension, since the patient has a β₁AR gene haplotype that predisposes to a favorable response to a treatment protocol including a beta blocker.

### Example 4

A patient with heart failure is receiving the beta blocker metoprolol, but has complaints of frequent dizzy spells upon standing from a seated or lying position. The patient's heart rate is found to be 55 in both the lying and standing positions, and the systolic and diastolic blood pressures decrease upon standing. The physician is concerned that the dizzy spells are due to the beta blocker, but also is cognizant that administration of a beta blocker is a recognized treatment for heart failure. A blood sample is obtained from the patient. Genomic DNA is isolated from leukocytes in the blood using standard phenol/chloroform precipitation techniques. The genomic DNA is used as a template for PCR amplification of the β₁AR gene or a fragment thereof covering the nucleotides where there are polymorphisms that define the haplotypes. The PCR product is purified and sequenced using an automated DNA sequencer and techniques known in the art. The nucleotides at each polymorphic site are identified and the haplotype is noted to be Haplotype 2. Because the patient has a β₁AR haplotype that corresponds to a high expressor, and because the beta blocker is the likely cause of the patient's dizzy spells in this instance, the dose of metoprolol is decreased by 50%.

### Example 5

A patient is being considered for inclusion in a clinical trial for a new beta blocker targeted for the treatment of individuals who are likely to respond to treatment with a beta blocker. A blood sample is obtained from the patient and genomic DNA is isolated from leukocytes in the blood sample using phenol/chloroform precipitation techniques known in the art. The genomic DNA is used as a template for amplification of the β₁AR gene or a fragment thereof by polymerase chain reaction (PCR) covering the nucleotides where the polymorphisms that define the haplotypes are located. The PCR product is purified and sequenced using an automated DNA sequencer. The nucleotides at each polymorphic site are identified and the haplotype is noted to be Haplotype 4. The clinician determines that administration of a beta blocker is indicated for the patient. The patient is selected for inclusion in the clinical trial.

### Example 6

A new beta blocker is synthesized and it is desirable to know how it interacts with the various human β₁AR haplotypes for the treatment of tachyarrhythmias. Mice are genetically engineered to express, individually, each of the 6 haplotypes. A beta agonist is given intravenously to the mice to increase the heart rate. Then, the beta blocker to be screened is administered. Mice with haplotype 1 fail to exhibit a decrease in heart rate after administration of the beta blocker. The clinician determines that variability in the response to the drug in the human population is likely, due to the genetic variation that comprises haplotype 1. Development of the drug is discontinued.

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method for determining whether a treatment protocol for a human patient who is suffering from one or more of heart failure, ischemic heart disease, cardiac arrhythmias, or hypertension comprises administration of a beta blocker, the method comprising:
(a) obtaining a biological sample from the patient;
(b) determining a β₁-adrenergic receptor (β₁AR) sequence of a β₁AR gene from the biological sample;
(c) identifying locations of any polymorphisms in the β₁AR sequence;
(d) assigning a haplotype to the β₁AR sequence based on the locations identified; and
(e) determining whether the treatment protocol comprises administration of a beta blocker to the patient based on the haplotype assigned.

2. The method of claim 1 wherein the haplotype is selected from the group consisting of haplotype 1, haplotype 2, haplotype 3, haplotype 4, haplotype 5, and haplotype 6.

3. The method of claim 2 wherein administration of a beta blocker is indicated in the treatment protocol for a patient having haplotype 2 or 4.

4. The method of claim 2 wherein administration of a beta blocker is not indicated in the treatment protocol for a patient having haplotype 1, 3, 5, or 6.

5. The method of claim 2 wherein the biological sample is blood, buccal swab, hair, tissue biopsy, or paraffin-embedded tissue.

6. The method of claim 2 wherein the beta blocker is selected from the group consisting of metoprolol, carvedilol, bucindolol, atenolol, bisoprolol, betaxolol, timolol, mebivolol, pindolol, propranolol and labetalol.

7. The method of claim 1 wherein the β₁AR sequence is determined from DNA or RNA in the biological sample.

8. A method for determining a suitable dose of a beta blocker as part of a treatment protocol for a human patient who is suffering from one or more of heart failure, ischemic heart disease, cardiac arrhythmias, or hypertension, the method comprising:
(a) obtaining a biological sample from the patient;
(b) determining a β₁-adrenergic receptor (β₁AR) sequence of a β₁AR gene from the biological sample;
(c) identifying locations of any polymorphisms in the β₁AR sequence;
(d) assigning a haplotype to the β₁AR sequence based on the locations identified; and
(e) determining a suitable dose of the beta blocker as part of the treatment protocol for the patient based on the haplotype assigned.

9. The method of claim 8 wherein the haplotype is selected from the group consisting of haplotype 1, haplotype 2, haplotype 3, haplotype 4, haplotype 5, and haplotype 6.

10. The method of claim 9 wherein the suitable dose for a patient having haplotype 2 or 4 is lower than a recommended dose.

11. The method of claim 9 wherein the suitable dose for a patient having haplotype 1, 3, 5, or 6 is higher than a recommended dose.

12 The method of claim 9 wherein the biological sample is blood, buccal swab, hair, tissue biopsy, or paraffin-embedded tissue.

13. The method of claim 9 wherein the beta blocker is selected from the group consisting of metoprolol, carvedilol, bucindolol, atenolol, bisoprolol, betaxolol, timolol, mebivolol, pindolol, propranolol and labetalol.

14. The method of claim 8 wherein the a β₁AR sequence is obtained from DNA or RNA in the biological sample.

15. A method for determining whether administration of a beta blocker to a patient is indicated or not indicated, the method comprising:
(a) obtaining a biological sample from the patient;
(b) determining a β₁-adrenergic receptor (β₁AR) sequence of a β₁AR gene from the biological sample;
(c) identifying locations of any polymorphisms in the β₁AR sequence;
(d) assigning a haplotype to the β₁AR sequence based on the locations identified; and
(e) determining that administration of a beta blocker is indicated or not indicated based on the haplotype assigned.

16. The method of claim 15, wherein the haplotype is selected from the group consisting of haplotype 1, haplotype 2, haplotype 3, haplotype 4, haplotype 5, and haplotype 6.

17. The method of claim 16, wherein administration is indicated in a patient having haplotype 2 or 4.

18. The method of claim 16 wherein administration is not indicated in a patient having haplotype 1, 3, 5, or 6.

19. The method of claim 15 wherein the biological sample is blood, buccal swab, hair, tissue biopsy, or paraffin-embedded tissue.

20. The method of claim 15 wherein the β₁AR sequence information is obtained from DNA or RNA in the biological sample.

21. The method of claim 15 wherein the method further comprises selecting candidate human subjects for participation in a clinical trial involving a beta blocker based on the determination of step (e).

22. A screening assay for evaluating the effectiveness of a beta blocker against one or more target β₁AR haplotypes comprising:
(a) establishing baseline expression levels for cells expressing one or more target β₁AR haplotypes;
(b) exposing cells that express one or more target β₁AR haplotypes to a beta blocker;
(c) determining expression levels of the one or more target β₁AR haplotypes;
(d) comparing the expression levels of step (c) with the baseline expression levels; and
(e) evaluating the effectiveness of the beta blocker against the one or more target β₁AR haplotypes based on the comparison of step (d).

23. The screening assay of claim 22 wherein the one or more target β₁AR haplotype is selected from the group consisting of haplotype 1, haplotype 2, haplotype 3, haplotype 4, haplotype 5, haplotype 6, and combinations thereof.

24. The screening assay of claim 22 wherein the assay is conducted in vitro, in vivo, or ex vivo.

25. The screening assay of claim 22 wherein the cells endogenously express the one or more target β₁AR haplotypes.

26. The screening assay of claim 22 wherein the cells are engineered to express the one or more target β₁AR haplotypes.
